(19) United States
(12) Patent Application Publication
(10) Pub. No.: US 2007/0253927 A1
(43) Pub. Date: Nov. 1, 2007

(54) Cosmetic compositions comprising at least one dielectrophile monomer and at least one radical initiator

(76) Inventors: Gwenaelle Jegou, Livry Gargan (FR); Nathalie Mougin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,921

(22) Filed: Apr. 13, 2007

Related U.S. Application Data
(60) Provisional application No. 60/796,875, filed on May 3, 2006.

(30) Foreign Application Priority Data
Apr. 13, 2006 (FR) 0603318

(51) Int. Cl. A61K 8/81

(52) U.S. Cl. 424/70.16; 424/63

(57) ABSTRACT

The present disclosure relates to novel compositions for the cosmetic treatment of keratinous fibers, such as the hair, comprising, in a cosmetically acceptable medium, at least one dielectrophile monomer and at least one radical initiator. The disclosure also relates to processes for the treatment of keratinous fibers comprising the composition. The composition in accordance with the present disclosure may makes it possible, when it is applied to keratinous fibers, such as the hair, to obtain a sheathing exhibiting an improved resistance to external agents.

This application claims benefit of U.S. Provisional Application No. 60/796,875, filed May 3, 2006, the contents of which are incorporated herein by reference. This application also claims benefit of priority under 35 U.S.C. §119 to French Patent Application No. FR 06/03318, filed Apr. 13, 2006, the contents of which are also incorporated herein by reference.

The present disclosure relates to novel compositions for the cosmetic treatment of keratinous fibers, such as the hair, comprising, in a cosmetically acceptable medium, at least one dielectrophile monomer and at least one radical initiator. The disclosure also relates to processes for the treatment of keratinous fibers starting from this composition and to its use in treating the fibers.

There exist numerous styling products which make it possible to contribute body, bulk or volume to the hair. One disadvantage related to these products, which can be based on film-forming polymers, lies in the fact that the cosmetic effect disappears after the first shampooing.

It is possible to envisage increasing the persistence of the deposit layer of polymers by directly carrying out a radical polymerization of certain monomers on the hair, as described in U.S. Pat. No. 5,362,486. Another alternative form is to treat the hair, after reduction of the keratin of the hair, by radical polymerization of monomers of methyl methacrylate (MMA, a monoelectrophile) or itaconic acid.

However, the treatments thus obtained are not very cosmetic and great damage to the fiber may be observed. The hair thus treated may be difficult to disentangle.

Furthermore, French Patent Application FR 2 833 489 discloses compositions for the treatment of the hair starting from compositions comprising electrophilic monomers capable of polymerizing anionically directly on the hair. These monomers, after polymerization, make it possible to obtain perfectly sheathed hairs. However, the sheathing obtained does not always exhibit satisfactory strength with respect to the various external assaults to which hair may be subjected.

There thus exists a need to find novel cosmetic compositions which make it possible to contribute body, bulk and/or volume to the hair and this in a lasting fashion, for example, in the face of the various assaults which the hair may be subjected to, such as shampooing, rubbing, light, bad weather, sweat and perming. It would be especially desirable to find novel cosmetic compositions that had these properties with regard to resistance to shampooing.

The present disclosure thus relates to a composition comprising, in a cosmetically acceptable medium, at least one dielectrophile monomer and at least one radical initiator.

Such a composition, when it is applied to keratinous fibers, such as the hair, makes it possible to obtain a sheathing exhibiting improved resistance to external agents while retaining completely separate hairs which can be styled without difficulty. Furthermore, the styling properties contributed to the fiber may be persistent. This is because there is thus formed, at the surface of the hairs, a protective sheathing which may be persistent towards one or more of the various attacks to which hair may be subjected, such as shampooing, rubbing, light, bad weather, sweat, sebum and perming. In at least one embodiment, the protective sheathing exhibits improved resistance of the coloring with regard to shampooing. The film formed is hydrophobic and can be mechanically resistant.

The sheathing obtained can be homogeneous and smooth and may possess excellent adhesion to the hair.

The polymer formed from the presently disclosed compositon coats the keratinous fiber, forming a sheathing on the fiber which may give it bulk, volume, gloss and softness.

The present disclosure also relates to a process for the treatment of keratinous fibers which comprises the application, to the keratinous fibers, of the composition of the disclosure and the use of the composition of the disclosure in the treatment of keratinous fibers, such as the hair.

The present disclosure further relates to a kit comprising, on the one hand, a composition comprising at least one dielectrophile monomer and, on the other hand, a second composition which comprises at least one radical initiator.

The at least one dielectrophile monomer used herein in is the monomer of formula (A): 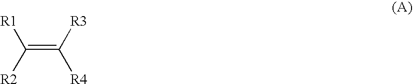
wherein:
R1 and R2 are chosen from, independently of one another, groups with little or no electron-withdrawing effect, (with little or no inductive withdrawing effect), such as:

hydrogen atoms,

saturated or unsaturated, linear, branched or cyclic hydrocarbon groups, for example, comprising from 1 to 20 carbon atoms, such as from 1 to 10 carbon atoms, optionally comprising at least one atom chosen from nitrogen, oxygen and sulphur atoms and optionally substituted by at least one group chosen from —OR, —C(O)OR, —C(O)R, —SR and halogen atoms,

modified and unmodified polyorganosiloxane residues, and

polyoxyalkylene groups,

wherein in at least one embodiment, R1 and R2 are hydrogen atoms, or, in at least one further embodiment, R1 is a hydrogen atom and R2 is a phenyl group optionally substituted by a halogen atom;

R3 and R4 are chosen from, independently of one another, electron-withdrawing groups, for example, chosen from —N(R)₃⁺, —S(R)₂⁺, —NO₂, —SO₂R, —C≡N, —C(O)OR, —C(O)SR, —C(O)NR₂, —F, —Cl, —Br, —I, —OR, —C(O)R and —SR groups, linear and branched alkenyl groups, linear and branched alkynyl groups, C₁-C₄ mono- and polyfluoroalkyl groups, aryl groups, such as phenyl, and aryloxy groups, such as phenoxyloxy;
   wherein R, which is identical or different, is chosen from hydrogen atoms and saturated or unsaturated, linear, branched or cyclic hydrocarbon groups, for example comprising from 1 to 20 carbon atoms, such as from 1 to 10 carbon atoms, optionally comprising at least one atom chosen from nitrogen, oxygen and sulphur atoms and optionally substituted by at least one group chosen from —OR′, —C(O)OR′, —C(O)R′, —SH, —SR′, —OH, halogen atoms, and residues of polymers which can be obtained by radical polymerization, by polycondensation and by ring opening, wherein R′ is chosen from C₁-C₁₀ alkyl groups;
      with the proviso that (A) cannot represent methyl 2-cyanoacrylate or itaconic acid.

As used herein, the term “electron-withdrawing group” is understood to mean a group which withdraws electrons by an inductive or inductive-withdrawing effect and, more specifically, any group which is more electronegative than carbon. Reference may be made to the work P R Wells, Prog. Phys. Org. Chem., 1968, Vol. 6, pp 111.

As used herein, the term “group with little or no electron-withdrawing effect” is understood to mean any group having an electronegativity less than or equal to that of carbon.

When monomer (A) is cyclic, the electron-withdrawing groups R3 and R4 are, in at least one embodiment, exocyclic, i.e., they do not form an integral part of the cyclic structure of the monomer.

The alkenyl and/or alkynyl groups mentioned above, in at least one embodiment, comprise from 2 to 20 carbon atoms, for example, from 2 to 10 carbon atoms.

In at least one embodiment, the saturated and unsaturated, linear, branched and cyclic hydrocarbon groups referred to above comprise, for example, from 1 to 20 carbon atoms and are chosen from cycloalkyl groups, aromatic groups and linear and branched alkyl, alkenyl and alkynyl groups, such as methyl, ethyl, n-butyl, tert-butyl, isobutyl, pentyl, hexyl, octyl, butenyl or butynyl groups.

In at least one further embodiment, substituted hydrocarbon groups useful herein are chosen from hydroxyalkyl and polyhaloalkyl groups.

Non-limiting examples of polymer residues as disclosed above include optionally modified hydrocarbonpolyorganosiloxanes. Mention may be made, for example, of polyalkylsiloxanes, such as polydimethylsiloxanes, polyarylsiloxanes, such as polyphenylsiloxanes, and polyarylalkylsiloxanes, such as polymethylphenylsiloxanes.

Among modified polyorganosiloxanes, non-limiting mention may be made of polydimethylsiloxanes comprising polyoxyalkylene and/or siloxy and/or silanol and/or amine and/or imine and/or fluoroalkyl groups.

Among polymer residues useful herein, non-limiting examples include polyoxyalkylene groups, such as polyoxyethylene groups and polyoxypropylene groups or copolymers thereof, having, in at least one embodiment, from 1 to 200 oxyalkylene units.

With respect to mono- or polyfluoroalkyl groups as referred to above, non-limiting examples include —(CH₂)ₙ(CF₂)ₘ—CF₃ and —(CH₂)ₙ—(CF₂)ₘ—CHF₂ wherein n=1-20 and m=1-20.

R1 to R4 can optionally be substituted by a group having a cosmetic activity. Groups having cosmetic activities include, for instance, groups having coloring, antioxidizing, UV-screening and/or conditioning functions.

Groups with a coloring function may be chosen, for example, from azo, quinone, methine, cyanomethine and triarylmethane groups.

Groups with an antioxidizing function may be chosen, for example, from groups of butylated hydroxyanisole (BHA), butylated hydroxytoluene (BHT) and vitamin E.

Groups with a UV-screening function may be chosen, for example, from benzophenone, cinnamate, benzoate, benzylidenecamphor and dibenzoylmethane groups.

Groups with a conditioning function may be chosen, for example, from cationic groups and fatty ester groups.

In at least one embodiment, the dielectrophile monomers of formula (A) are chosen from:
the derivatives benzylidenemalononitrile (1), 2-(4-chlorobenzylidene)malononitrile (2), ethyl 2-cyano-3-phenylacrylate (3) and ethyl 2-cyano-3-(4-chlorophenyl)acrylate (4), described in Sayyah, J. Polymer Research, 2000, p 97; 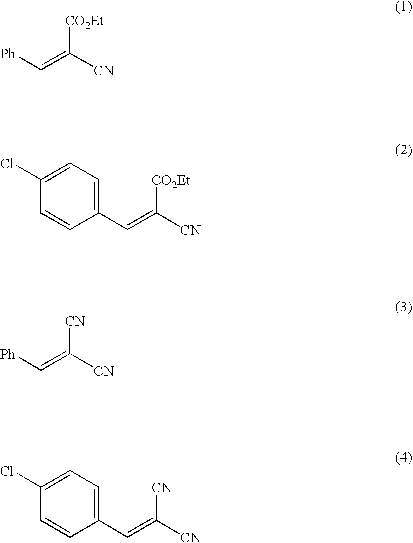
methylidenemalonate derivatives, such as:
   diethyl 2-methylenemalonate (5), described by Hopff, Makromoleculare Chemie, 1961, p 95, De Keyser, J. Pharm. Sci., 1991, p 67 and Klemarczyk, Polymer, 1998, p 173; 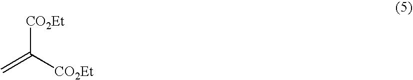
   C₁-C₈-alkyl carboxyacrylate (5′), described by Martinez J M. in Patent Application WO 9749436; and 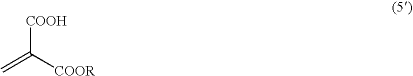
   ethyl 2-ethoxycarbonylmethyloxycarbonylacrylate (6), described by Breton, Biomaterials, 1998, p 271 and Couvreur, Pharmaceutical Research, 1994, p 1270; 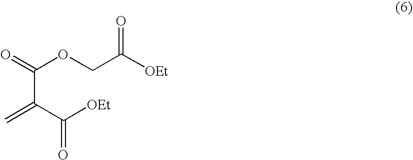
the derivatives methyl α-(methylsulphonyl)acrylate (7), ethyl α-(methylsulphonyl)acrylate (8), methyl α-(tert-butylsulphonyl)acrylate (9), tert-butyl α-(methylsulphonyl)acrylate (10) and tert-butyl α-(tert-butylsulphonyl)acrylate (11), described by Gipstein, J.Org.Chem., 1980, p 1486, and the derivatives 1,1-bis(methylsulphonyl)ethylene (12), 1-acetyl-1-(methylsulphonyl)ethylene (13), methyl a-(methylsulphonyl)vinylsulphonate (14) and α-(methylsulphonyl)acrylonitrile (15), described by Shearer, U.S. Pat. No. 2,748,050; and 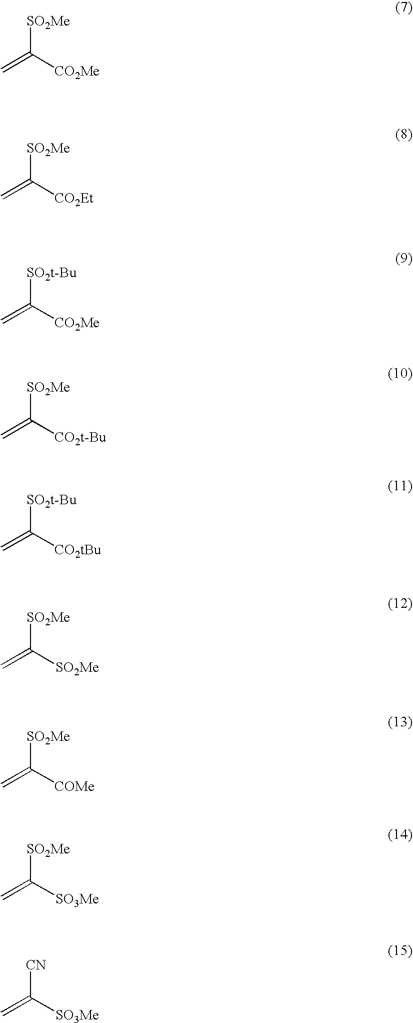
itaconate and itaconimide derivatives, other than itaconic acid, such as:
   dimethyl itaconate (16), described by Bachrach, European Polymer Journal, 1976, p 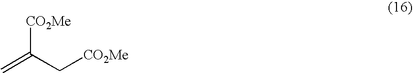
   N-butylitaconimide (17), N-(4-tolyl)itaconimide (18), N-(2-ethylphenyl)itaconimide (19) and N-(2,6-diethylphenyl)itaconimide (20), described by Wanatabe, J.Polymer Science: Part A: Polymer Chemistry, 1994, p 2073;

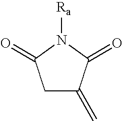

Rₐ═Bu (17), 4-tolyl (18), 2-ethylphenyl (19), 2,6-diethylphenyl (20)

In at least one embodiment, monomer (A) is chosen from cyanoacrylates of formula (B) and derivatives thereof:

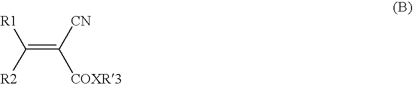

wherein X is chosen from NH, S and O,

R1 and R2 are as defined in formula (A), and in one embodiment, both R1 and R2 are hydrogen atoms, and

R′3 is R as defined in formula (A).

In at least one embodiment, X is O.

In at least one further embodiment, R′3 is chosen from linear and branched alkyl radicals comprising from 6 to 10 carbon atoms, and alkenyl radicals comprising from 6 to 10 carbon atoms.

In at least one embodiment, the monomers of formula (B) are chosen from:

a) the family of polyfluoroalkyl 2-cyanoacrylates, such as:

2,2,3,3-tetrafluoropropyl ester of 2-cyano-2-propenoic acid of formula (D):

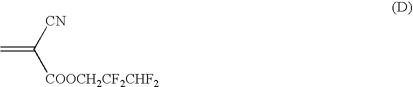

and 2,2,2-trifluoroethyl ester of 2-cyano-2-propenoic acid of formula (E):

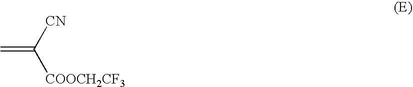

b) alkyl and alkoxyalkyl 2-cyanoacrylates of formula (C):

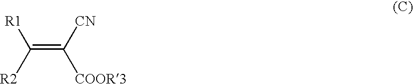

wherein R′3 is chosen from C₁-C₁₀ alkyl radicals, C₂-C₁₀ alkenyl radicals and (C₁-C₄)alkoxy(C₁-C₁₀)alkyl radicals;

for example, ethyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, isoamyl cyanoacrylate, allyl 2-cyanoacrylate and methoxypropyl 2-cyanoacrylate; and

c) benzylidenemalononitrile derivative of formula (1) and 2-(4-chlorobenzylidene)malononitrile derivative of formula (2) and mixtures thereof 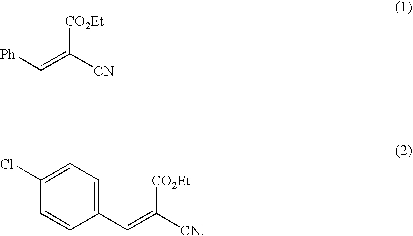
In at least one embodiment, the cyanoacrylate monomers of formula (C) are used, and in a further embodiment, said monomers are chosen from C₆-C₁₀ alkyls and C₆-C₁₀ alkenyl cyanoacrylates.

In at least one further embodiment, the monomers of formula (C) are chosen from octyl cyanoacrylates of formula (F) and mixtures thereof: 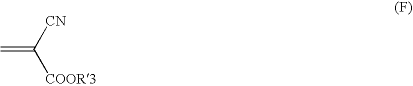
wherein R′3 is chosen from:
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

The cyanoacrylate monomer (C), in at least one embodiment, is chosen from linear and branched octyl 2-cyanoacrylates sold under the commercial reference Ritelok CDN 1064 by Chemence.

The monomers used in accordance with the disclosure can be covalently attached to supports, such as polymers, oligomers or dendrimers. The polymer or oligomer can be linear, branched, of comb structure or of block structure. The distribution of the monomers of the disclosure over the polymer, oligomer or dendrimer structure can be random, in the end position or in the form of blocks.

The cyanoacrylate monomers of formula (B) according to the present disclosure can be synthesized according to known methods described in the art. For example, the cyanoacrylate monomers can be synthesized according to the teaching of U.S. Pat. Nos. 3,527,224, 3,591,767, 3,667,472, 3,995,641, 4,035,334 and 4,650,826.

The at least one dielectrophile monomer, for example, the cyanoacrylate monomer of formula (B), such as octyl 2-cyanoacrylate, can be present in an amount ranging from 0.1 to 99% by weight, with respect to the total weight of the composition comprising it.

In at least one embodiment, the amount of cyanoacrylate monomers present ranges from 0.1 to 80% by weight, relative to the total weight of the composition, for example, from 1 to 50% by weight.

In the context of the present disclosure, the term “radical polymerization” is understood to mean any polymerization initiated by a radical generator or initiator.

The radical initiator may be soluble in water or in organic solvents.

The reactivity of a radical generator or initiator is often given by its half life at different temperatures or by its half life temperature at different times.

The half life at different temperatures can be calculated via the following formula:
t_{1/2}=(In2)/kd

wherein kd=A·e^{-(Ea/RT)},
Ea=123.80 kJ/mol,
R=8.3142 J/mol·K
A=1.62E⁺¹⁴s⁻¹ and
T in degrees Kelvin (K) corresponds to the temperature in degrees Celsius (° C) with the addition of 273.15.

The at least one radical initiator disclosed herein can be an initiator of peroxide type, for example, of formula (I) or (II), of azo type, of persulphate type, of redox type or mixtures thereof:
R₁—O—O—R₂ (I)
R₁—O—O—R₃—O—O—R₂ (II)
wherein:
R₁ and R₂, which are identical or different, are chosen from hydrogen atoms and groups chosen from optionally substituted aryls, linear and branched (C₁-C₆)alkyls, acyls R′C(O)— and —C(O)R″, and esters R′OC(O)— and —(O)COR″, wherein R′ and R″, which are identical or different, are chosen from linear and branched (C₁-C₆)alkyls and optionally substituted aryl groups;
R₃ is a divalent alkylene radical —(CR₄R₅)_{q}— wherein R₄ and R₅, which are identical or different, are chosen from hydrogen and halogen atoms, and linear and branched (C₁-C₆)alkyl groups, wherein q is an integer from 1 to 6.

The term “optionally substituted aryl” radical as used herein means a fused or nonfused mono- or polycyclic group comprising from 6 to 22 carbon atoms, at least one ring of which is aromatic; the aryl radical is, for example, a phenyl, biphenyl, naphthyl, indenyl, anthracenyl or tetrahydronaphthyl; this group optionally being substituted by a radical chosen from:
C₁-C₁₆ alkyls, for example C₁-C₈ alkyls;
halogen atoms, such as chlorine, fluorine or bromine;
hydroxyl groups;
C₁-C₂ alkoxy radicals;
C₂-C₄ (poly)hydroxyalkoxy radicals;
amino radicals optionally substituted by one or two identical or different C₁-C₆ alkyl radical;
acylamino radicals of the formula —NR₆—C(O)R₇ wherein R₆ and R₇, which are identical or different, are chosen from hydrogen atoms and C₁-C₄ alkyl radicals;
carbamoyl radicals of the formula R₆R₇N—C(O)— wherein R₆ and R₇, which are identical or different, are chosen from hydrogen atoms and C₁-C₄ alkyl radicals;
alkylsulphonylamino radicals of the formula R₆SO₂—NR₇— wherein R₆ and R₇, which are identical or different, are chosen from hydrogen atoms and C₁-C₄ alkyl radicals;
aminosulphonyl radicals of the formula R₆R₇N—SO₂— wherein R₆ and R₇, which are identical or different, are chosen from hydrogen atoms and C₁-C₄ alkyl radicals;
carboxyl radicals in acid or salified form (for example, salified with an alkali metal or a substituted or unsubstituted ammonium);
cyano groups (CN); and
polyhaloalkyl groups, such as C₁-C₆ trihaloalkyls, for example, trifluoromethyl.

Non-limiting mention may be made, among the peroxides of formula (I), of thehydroperoxides of formula R₁—O—O—H, such as benzoyl peroxide, acetyl peroxide, lauryl peroxide and decanoyl peroxide. In at least one embodiment, the peroxides may be chosen from commercial peroxides, such as those sold by Arkema under the following names:

| | | 10 h Decomposition |
|---|---|---|
| | | temperature (t_{1/2}) |
| Luperox ® TBH70 X | tert-Butyl hydroperoxide | 170-172° C., benzene |
| | | 164° C., |
| | | chlorobenzene |
| Luperox ® TAH85 | tert-Amyl hydroperoxide | |
| Luperox ® CU80 | Cumyl hydroperoxide | 158° C., benzene |
| | | 140° C., |
| | | chlorobenzene |
| Luperox ® 2,5-2,5 | 2,5-Dimethyl-2,5-di- | |
| | (hydroperoxy)hexane | |
| Luperox ® DH | Diisopropylbenzene | |
| | monohydroperoxide | |
| Luperox ® PMHP | para-Menthane | |
| | hydroperoxide | |

In at least one embodiment, the initiator is chosen from dialkyl peroxides of formula (I), such as dicumyl peroxide and commercial peroxides, for example, sold by Arkema under the following names:

| | | 10 h Decomposition |
|---|---|---|
| | | temperature (t_{1/2}) |
| Luperox ® 130 | 2,5-Dimethyl-2,5-di(tert- | 131° C., benzene |
| | butylperoxy)hex-3-yne | |
| Luperox ® DI | Di(tert-butyl) peroxide | 129° C., benzene |
| Luperox ® DTA | Di(tert-amyl) peroxide | 123° C., benzene |
| Luperox ® 101 | 2,5-Dimethyl-2,5-di(tert- | 120° C., benzene |
| | butylperoxy)hexane | |
| Luperox ® DC | Dicumyl peroxide | 115° C., benzene |
| Luperox ® DCSC | | |

In another embodiment, the peroxide initiator of formula (I) is chosen from diacyl peroxides of formula R′—C(O)—O—O—C(O)—R″, such as those sold, for example, by Arkema under the following names:

| | | 10 h Decomposition |
|---|---|---|
| | | temperature (t_{1/2}) |
| Luperox ® A75 | Benzoyl peroxide | 70-73° C., benzene |
| Luperox ® LP | Lauroyl peroxide | 65° C., benzene |
| Luperox ® DEC | Decanoyl peroxide | 61° C., benzene |
| Luperox ® 219M75 | 3,5,5-Trimethylhexanoyl | |
| | peroxide | |
| Luperox ® 219EN50 | 3,5,5-Trimethylhexanoyl | |
| | peroxide | |

In at least one embodiment, the peroxide initiator of formula (I) is chosen from peroxy esters of formula R′—C(O)—O—O—R₂, such as t-butyl peroxybenzoate, t-butyl peroxypivalate (Lupersol™ 1, Atochem), t-butyl peroxy-2-ethylhexanoate (TrigonoX™ 21-C50, Akzo Chemicals Inc.) and those sold by Arkema under the following names:

| | | 10 h |
|---|---|---|
| | | Decomposition |
| | | temperature (t_{1/2}) |
| Luperox ® P | tert-Butyl peroxybenzoate | 104° C., benzene |
| Luperox ® 7M50 | tert-Butyl peroxyacetate | 102° C., benzene |
| Luperox ® 270 | tert-Butyl peroxy-3,5,5- | |
| | trimethylhexanoate | |
| Luperox ® 570 | tert-Amyl peroxy-3,5,5- | |
| | trimethylhexanoate | |
| Luperox ® 80M75 | tert-Butyl peroxyisobutyrate | 82° C., benzene |
| Luperox ® 26 | tert-Butyl peroxy-2- | 58° C., benzene |
| | ethylhexanoate | |
| Luperox ® 575 | tert-Amyl peroxy-2- | 75° C., benzene |
| | ethylhexanoate | |
| Luperox ® 11M75 | tert-Butyl peroxypivalate | 58° C., benzene |
| Luperox ® 554M75 | tert-Amyl peroxypivalate | 55° C., benzene |
| Luperox ® 10 | tert-Butyl peroxyneodecanoate | 48° C., benzene |
| Luperox ® 546M75 | tert-Amyl peroxyneodecanoate | 46° C., benzene |
| Luperox ® 188M70 | Cumyl peroxyneodecanoate | 38° C., benzene |
| Luperox ® | 3-Hydroxy-1,1-di-methylbutyl | |
| 610EN50 | peroxyneo-decanoate | |

In at least one further embodiment, the peroxide initiator of formula (I) is chosen from monoperoxycarbonates of formula R′—O—C(O)—O—O—R₂, such as those sold by Arkema under the following names:

| | | 10 h |
|---|---|---|
| | | Decomposition |
| | | temperature (t_{1/2}) |
| Luperox ® | OO-tert-Butyl O-isopropyl | 99° C., benzene |
| TBICM75 | monoperoxycarbonate | |
| Luperox ® TBEC | OO-tert-Butyl O-(2-ethylhexyl) | 100° C., benzene |
| | monoperoxycarbonate | |
| Luperox ® TAEC | OO-tert-Amyl O-(2-ethylhexyl) | 117° C., benzene |
| | monoperoxycarbonate | |
| Luperox ® | Poly(tert-butyl peroxycarbonate) | 119° C., benzene |
| JWEB50 | | |

In at least one embodiment, the peroxide initiator of formula (I) is chosen from peroxydicarbonates of formula R′—O—C(O)—O—O—C(O)—O—R″, such as diacetyl peroxydicarbonate, di(4-(t-butyl)cyclohexyl) peroxydicarbonate (Perkadox™ 16S, Akzo Chemicals), di(2-ethylhexyl) peroxydicarbonate and di(2-ethylhexyl) peroxydicarbonate (Luperox® 223).

In at least one further embodiment, the peroxide initiator of formula (I) is chosen from peroxyketals of formula R₁—O—O—CR₄R₅—O—O—R₂, such as those sold by Arkema under the following names:

| | | 10 h |
|---|---|---|
| | | Decomposition |
| | | temperature (t_{1/2}) |
| Luperox ® 331 | Di(tert- | 97° C. dodecane |
| | butylperoxy)cyclohexane | |
| Luperox ® 233M50 | Ethyl 3,3-di(tert- | 114° C. dodecane |
| | butylperoxy)butyrate | |
| Luperox ® 533M65 | Ethyl 3,3-di(tert- | 112° C. dodecane |
| | amylperoxy)butyrate | |
| Luperox ® 230M50 | n-Butyl 4,4-di(tert- | |
| | butylperoxy)valerate | |
| Luperox ® 220M50 | 2,2-Di(tert-butylperoxy)butane | |
| Luperox ® 231M50 | 1,1-Di(tert-butylperoxy)-3,3,5- | 96° C. dodecane |
| | trimethylcyclohexane | |
| Luperox ® 531M60 | 1,1-Di(tert-amylperoxy)- | 93° C. dodecane |
| | cyclohexane | |

In at least one embodiment, the initiator is chosen from initiators which are soluble in organic solvents, for example, the following azo initiators:

| | | 10 h Decomposition |
|---|---|---|
| | | temperature (t_{1/2}) |
| 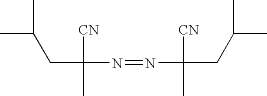 | Vazo ® 52: 2,2′-azobis(2,4- dimethylvaleronitrile) from DuPont Chemicals | 52° C., toluene |
| 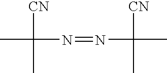 | Vazo ® 64: 2,2′- azobis(isobutyronitrile) from DuPont Chemicals | 64-65° C., toluene |
| 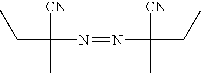 | Vazo ® 67: 2,2′-azobis(2- methylbutyronitrile) from DuPont Chemicals (Vazo ™ 67 from DuPont Chemicals) | 66° C., chlorobenzene |
| 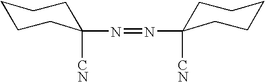 | Vazo ® 88: 1,1′-azobis(1- cyclohexanecarbonitrile) from DuPont Chemicals, | 88° C., toluene |

and 2,2′-Azobis(4-methoxy-2,4-dimethylvaleronitrile) (Vazo™ 33 from DuPont Chemicals) and 2,2′-azobis(methyl isobutyrate) (V-601 from Wako).

In at least one further embodiment, the initiator is chosen from water-soluble azo initiators, for example:

| | | 10 h |
|---|---|---|
| | | Decomposition |
| Commercial | | temperature |
| code | Formula of the water-soluble initiator | (t_{1/2}) |
| VA-041 from Wako | 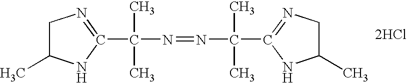 | 41° C. |
| VA-044 from Wako | 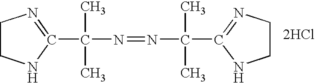 | 44° C. |
| VA-046B from Wako | 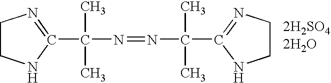 | 47° C. |
| VA-057 from Wako | 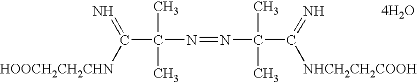 | 57° C. |
| VA-058 from Wako | 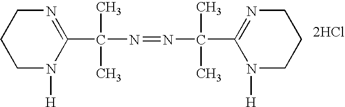 | 58° C. |
| VA-060 from Wako | 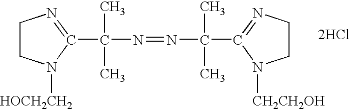 | 60° C. |
| VA-061 from Wako | 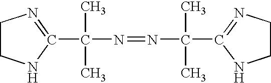 | 61° C. |
| VA-080 from Wako | 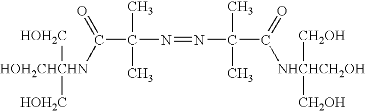 | 80° C. |
| VA-085 from Wako | 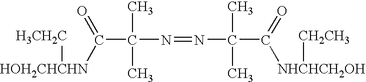 | 85° C. |
| VA-085 from Wako | 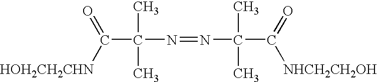 | 87° C. |
| Vazo ® 68 WSP from DuPont Chemicals | 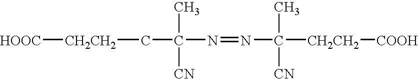 | |
| Vazo ® 56 WSP and 56 WSW from DuPont or V-50 ™ from Wako | 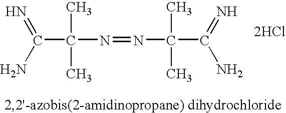 | 57° C. |

The initiator may be activated either photochemically or thermally.

In at least one embodiment, the initiator is chosen from persulphate initiators, for example, potassium persulphate (decomposition temperature: 10 h (t_{1/2})=60° C. in water), sodium persulphate and ammonium persulphate.

In at least one further embodiment, the initiator is chosen from combinations of persulphate and of reducing agents as redox initiators, such as sodium metabisulphite or sodium bisulphite, and also from systems based on peroxides and on tertiary amines (for example the pairs: benzoyl peroxide plus dimethylaniline), and systems based on hydroperoxides and on transition metals, such as the cumene hydroperoxide plus cobalt naphthenate mixture.

In at least one embodiment, the initiator is chosen from photoinitiators capable of being activated by UV irradiation at absorption wavelengths from 250 nm to 450 nm and, for example, greater than 351 nm, for example, benzoin ethers, such as benzoin methyl ether, benzoin isopropyl ether, substituted benzoin ethers, arylphosphine oxides, substituted acetophenone, such as 2,2-dimethoxy-2-phenylacetophenone, and substituted α-ketol (α-hydroxyketones).

In at least one further embodiment, the initiator is chosen from commercially available photoinitiators, for example, Irgacure™ 819 and Darocur™ 1173 (available from Ciba-Geigy Corp., Hawthorne, N.Y.), Lucern TPO™ (available from BASF, Parsippany, N.J.) and Irgacure™ 651 (2,2-dimethoxy-1,2-diphenyl-1-ethanone), available from Ciba-Geigy corporation.

The choice of the initiator will be guided according to the kinetics of decomposition and thus according to its decomposition temperature, the nature of the decomposition agents, its solubility, and the like.

Thus, a person skilled in the art will choose the initiator according to the desired application temperature and the desired reaction duration.

At least one embodiment of the present disclosure relates to a composition comprising at least one initiator having a 10 h decomposition temperature (t_{1/2}) from 45 C. to 180° C., for example, from 45° C. to 120° C.

The at least one radical initiator is present in an amount ranging from 0.01% to 40% by weight of the at least one dielectrophile monomer, for example, from 0.1% to 35%, such as from 1% to 30% by weight, relative to the weight of the monomer.

The polymerization can optionally be assisted by heating the monomer in the presence of the initiator, the temperature applied varying from 20° C. to 100° C. and, for example, from 20° C. to 90° C.

The cosmetically acceptable medium disclosed herein may comprise at least one liquid organic solvent, and may further comprise water. In at least one embodiment, the medium is anhydrous, i.e., comprising less than 1% by weight of water with respect to the total weight of the composition.

The cosmetically acceptable medium of the composition of the disclosure can be used homogeneously, can be provided in the form of an emulsion or can be encapsulated. The dispersed or continuous phase of the emulsion can then comprise water, C₁-C₄ aliphatic alcohols or mixtures thereof. The capsules or microcapsules comprising the composition of the disclosure can be dispersed in an anhydrous medium as defined above, water, C₁-C₄ aliphatic alcohols or mixtures thereof.

The cosmetically acceptable medium of the compositions of the disclosure can comprise at least one liquid organic solvent, wherein the solvent is other than the cyanoacrylate monomer.

As used herein, the term “organic solvent” is understood to mean an organic substance capable of dissolving another substance without chemically modifying it.

The organic solvents useful herein may be chosen from compounds which are liquid at a temperature of 25° C. and at 105 Pa (760 mmHg).

The organic solvent may be chosen, for example, from aromatic alcohols, such as benzyl alcohol; liquid fatty alcohols, such as C₁₀-C₃₀ fatty alcohols; modified and unmodified polyols, such as glycerol, glycol, propylene glycol, dipropylene glycol, butylene glycol and butyl diglycol; volatile silicones, such as cyclopentasiloxane, cyclohexasiloxane, polydimethylsiloxanes modified and unmodified by alkyl and/or amine and/or imine and/or fluoroalkyl and/or carboxyl and/or betaine and/or quaternary ammonium functional groups; liquid modified polydimethylsiloxanes; mineral, organic and vegetable oils; alkanes, such as C₅ to C₁₀ alkanes; liquid fatty acids; and liquid fatty esters, such as liquid fatty alcohol benzoates and salicylates.

In at least one embodiment, the organic solvent is chosen from organic oils; silicones, such as volatile silicones, silicone gums and oils which are or are not aminated and mixtures thereof; mineral oils; vegetable oils, such as olive oil, castor oil, rapeseed oil, coconut oil, wheat germ oil, sweet almond oil, avocado oil, macadamia oil, apricot oil, safflower oil, candlenut oil, camelina oil, tamanu oil and lemon oil; and also organic compounds, such as C₅-C₁₀ alkanes, acetone, methyl ethyl ketone, liquid esters of C₁-C₂₀ acids and of C₁-C₈ alcohols, such as methyl acetate, butyl acetate, ethyl acetate and isopropyl myristate, dimethoxyethane, diethoxyethane, liquid C₁₀-C₃₀ fatty alcohols, such as oleyl alcohol, liquid C₁₀-C₃₀ fatty alcohol esters, such as C₁₀-C₃₀ fatty alcohol benzoates, and mixtures thereof; polybutene oil, isononyl isononanoate, isostearyl malate, pentaerythrityl tetraisostearate, tridecyl trimelate, the cyclopentasiloxane (14.7% by weight)/polydimethylsiloxane dihydroxylated in the α and ω positions (85.3% by weight) mixture, and the mixtures thereof.

In at least one embodiment, the organic solvent is composed of a silicone or a silicone mixture, such as polyalkylsiloxanes and, for example, liquid polydimethylsiloxanes and liquid modified polydimethylsiloxanes, the viscosity of the silicone and/or of the mixture of silicones at 25° C. being from 0.1 cSt to 1,000,000 cSt, such as from 1 cSt to 30,000 cSt. In at least one further embodiment, the silicone can be chosen from cycloalkylsiloxanes.

In at least one embodiment, the organic solvent is chosen from the following oils:

the α,ω-dihydroxylated polydimethylsiloxane/cyclopentadimethylsiloxane (14.7/85.3) mixture sold by Dow Corning under the name of DC 1501 Fluid;

the α,ω-dihydroxylated polydimethylsiloxane/polydimethylsiloxane mixture sold by Dow Corning under the name of DC 1503 Fluid;

the dimethicone/cyclopentadimethylsiloxane mixture sold by Dow Corning under the name of DC 1411 Fluid or that sold by Bayer under the name SF1214;

the cyclopentadimethylsiloxane sold by Dow Corning under the name of DC245 Fluid;

and the respective mixtures of these oils.

The at least one liquid organic solvent of the composition can be present in an amount ranging from 0.01 to 99% by weight, for example, from 50% to 99% by weight, with respect to the total weight of the composition.

The cosmetic composition according to the disclosure can additionally comprise at least one pigment.

The use of a pigment in the cosmetic composition in accordance with the disclosure may make it possible to obtain visible colorings, for example, on dark hair, since the surface pigment masks the natural color of the fiber.

The composition in accordance with the disclosure thus may exhibit the advantage of colorings which exhibit good resistance to the various assaults to which the hair may be subjected, such as fatty substances or shampoos.

Furthermore, the cosmetic composition according to the present disclosure may make it possible to result in visible and highly chromatic colorings on a keratinous fiber, for example, a dark fiber, without it being necessary to lighten or bleach the keratinous fibers and consequently without physical damage to the keratinous fibers.

Within the meaning of the present disclosure, the term “pigment” is understood to mean any organic and/or inorganic entity having a solublity in water of less than 0.01% at 20° C., for example, at less than 0.0001%, and exhibiting an absorption in a range from 350 to 700 nm, for example, an absorption with a maximum.

The pigments used in the composition according to the disclosure can be chosen, for example, from known organic and/or inorganic pigments of the art, such as those which are described in Kirk-Othmer's Encyclopedia of Chemical Technology and in Ullmann's Encyclopedia of Industrial Chemistry.

These pigments can be provided in the form of a powder or of a pigment paste. They can be coated or uncoated.

The pigments in accordance with the present disclosure can, for example, be chosen from white and colored pigments, lakes, special effect pigments, such as pearlescent agents or glitter, and mixtures thereof.

In at least one embodiment, pigments may be chosen from white and colored inorganic pigments, for example, titanium dioxide, which is or is not surface treated, zirconium and cerium oxides, iron and chromium oxides, manganese violet, ultramarine blue, chromium hydrate and ferric blue. For example, the following inorganic pigments can be used: Ta₂O₅, Ti₃O₅, Ti₂O₃, TiO, ZrO₂ as a mixture with TiO₂, ZrO₂, Nb₂O₅, CeO₂, and ZnS.

In at least one further embodiment, white and colored organic pigments may be chosen from nitroso, nitro, azo, xanthene, quinoline, anthraquinone and phthalocyanine compounds, compounds of metal complex type, and isoindolinone, isoindoline, quinacridone, perinone, perylene, diketopyrrolopyrrole, thioindigo, dioxazine, triphenylmethane and quinophthalone compounds.

In at least one embodiment, white and colored organic pigments can be chosen from carmine, carbon black, aniline black, azo yellow, quinacridone, phthalocyanine blue, sorghum red, the blue pigments codified in the Color Index under the references Cl 42090, 69800, 69825, 73000, 74100 and 74160, the yellow pigments codified in the Color Index under the references Cl 11680, 11710,15985, 19140, 20040, 21100, 21108, 47000 and 47005, the green pigments codified in the Color Index under the references Cl 61565, 61570 and 74260, the orange pigments codified in the Color Index under the refences Cl 11725, 15510, 45370 and 71105, the red pigments codified in the Color Index under the references Cl 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole and phenol derivatives, as are described in French Patent FR 2 679 771.

In at least one further embodiment, the pigment may be chosen from pigment pastes formed of organic pigment, such as the products sold by Hoechst under the name:

Cosmenyl Yellow 10G: Pigment Yellow 3 (Cl 11710);

Cosmenyl Yellow G: Pigment Yellow 1 (Cl 11680);

Cosmenyl Orange GR: Pigment Orange 43 (Cl 71105);

Cosmenyl Red R: Pigment Red 4 (Cl 12085);

Cosmenyl Carmine FB: Pigment Red 5 (Cl 12490);

Cosmenyl Violet RL: Pigment Violet 23 (Cl 51319);

Cosmenyl Blue A2R: Pigment Blue 15.1 (Cl 74160);

Cosmenyl Green GG: Pigment Green 7 (Cl 74260); and

Cosmenyl Black R: Pigment Black 7 (Cl 77266).

The pigments in accordance with the present disclosure can also be in the form of composite pigments, as are described in European Patent EP 1 184 426. These composite pigments can be composed, for example, of particles comprising an inorganic core, at least one binder, which provides for the attachment of the organic pigments to the core, and at least one organic pigment which at least partially covers the core.

The term “lake” is understood to mean dyes adsorbed onto insoluble particles, the combination thus obtained remaining insoluble during use. The inorganic substrates onto which the dyes are adsorbed are, for example, alumina, calcium sodium borosilicate, calcium aluminium borosilicate and aluminium. Mention may be made, among organic pigments, of cochineal carmine.

Examples of lakes useful herein include but are not limited to the products known under the following names: D & C Red 21 (Cl 45 380), D & C Orange 5 (Cl 45 370), D & C Red 27 (Cl 45 410), D & C Orange 10 (Cl 45 425), D & C Red 3 (Cl 45 430), D & C Red 7 (Cl 15850:1), D & C Red 4 (Cl 15510), D & C Red 33 (CI 17200), D & C Yellow 5 (Cl 19 140), D & C Yellow 6 (Cl 15 985), D & C Green (Cl 61 570), D & C Yellow 10 (Cl 77 002), D & C Green 3 (Cl 42 053) and D & C Blue 1 (Cl 42 090).

As used herein, the term “special effect pigments” is understood to mean pigments which can create a colored appearance (characterized by a certain hue, a certain vividness and a certain lightness) which is not uniform and which changes as a function of the conditions of observation (light, temperature, angles of observation, and the like). They thereby contrast with white or colored pigments, which provide a conventional opaque, semitransparent or transparent uniform color.

In at least one embodiment, the special effect pigments are chosen from white pearlescent pigments, such as mica covered with titanium dioxide or with bismuth oxychloride, colored pearlescent pigments, such as mica covered with titanium dioxide and with iron oxides, mica covered with titanium dioxide and, for example, with ferric blue or with chromium oxide or mica covered with titanium dioxide and with an organic pigment as defined above, and pearlescent pigments based on bismuth oxychloride. In at least one further embodiment, pearlescent pigments may be chosen from: Cellini sold by Engelhard (mica-TiO₂-lake), Prestige sold by Eckart (mica-TiO₂), Prestige Bronze sold by Eckart (mica-Fe₂O₃) or Colorona sold by Merck (mica-TiO₂—Fe₂O₃).

In at least one embodiment, pigments may be chosen from pigments with an interference effect which are not attached to a substrate, such as liquid crystals (Helicones HC from Wacker), and interference holographic glitter (Geometric Pigment or Spectra f/x from Spectratek). Special effect pigments also comprise fluorescent pigments, whether they are substances which are fluorescent in daylight or which produce ultraviolet fluorescence, phosphorescent pigments, photochromic pigments, thermochromic pigments and quantum dots, for example sold by Quantum Dots Corporation.

Quantum dots are luminescent semiconductor nanoparticles capable of emitting, under light excitation, radiation exhibiting a wavelength ranging from 400 nm to 700 nm. These nanoparticles are known from the literature. For example, they can be manufactured according to the processes described in U.S. Pat. Nos. 6,225,198 or 5,990,479, in the publications which are cited therein and in the following publications: Dabboussi B. O. et al., “(CdSe)ZnS core-shell quantum dots: synthesis and characterisation of a size series of highly luminescent nanocristallites”, Journal of Physical Chemistry B, vol. 101, 1997, pp 9463-9475, and Peng, Xiaogang et al., “Epitaxial growth of highly luminescent CdSe/CdS core/shell nanocrystals with photostability and electronic accessibility”, Journal of the American Chemical Society, vol.119, No. 30, pp 7019-7029.

The variety of the pigments which can be used in the present disclosure makes it possible to obtain a rich palette of colors as well as specific optical effects, such as interference, and metallic effects.

According to at least one embodiment, the pigments are colored pigments. As used herein, the term “colored pigments” is understood to mean pigments other than white pigments.

The size of the pigment of use in the context of the present disclosure can be in a range from 10 nm to 200 μm, such as from 20 nm to 80 μm and, for example, from 30 nm to 50 μm.

The pigments can be coated with organic or inorganic compounds.

The organic agent with which the pigments are treated can be deposited on the pigments by evaporation of solvent, chemical reaction between the molecules of the surface agent and/or creation of a covalent bond between the surface agent and the pigments or fillers.

Within the meaning of the present disclosure, the surface treatment is such that a surface-treated pigment retains its intrinsic pretreatment pigment properties.

The surface treatment can thus be carried out, for example, by chemical reaction of a surface agent with the surface of the pigments and creation of a covalent bond between the surface agent and the pigments. This method is described, for example, in U.S. Pat. No. 4,578,266.

In at least one embodiment, the pigment will be made of an organic agent covalently bonded to the pigments.

The agent for the surface treatment can be present in an amount ranging from 0.1 to 50% by weight, such as from 0.5 to 30% by weight and for example, from 1 to 10% by weight of the total weight of the surface-treated pigments or fillers.

In at least one embodiment, the surface treatments of the pigments are chosen from the following treatments:

a PEG-Silicone treatment, such as the AQ surface treatment marketed by LCW;

a Chitosan treatment, such as the CTS surface treatment marketed by LCW;

a Triethoxycaprylylsilane treatment, such as the AS surface treatment sold by LCW;

a Methicone treatment, such as the SI surface treatment marketed by LCW;

a Dimethicone treatment, such as the Covasil 3.05 surface treatment marketed by LCW;

a Dimethicone/Trimethylsiloxysilicate treatment, such as the Covasil 4.05 surface treatment marketed by LCW;

a Lauroyl Lysine treatment, such as the LL surface treatment marketed by LCW;

a Lauroyl Lysine Dimethicone treatment, such as LL/SI surface treatment marketed by LCW;

a Magnesium Myristate treatment, such as the MM surface treatment marketed by LCW;

an Aluminium Dimyristate treatment, such as the Ml surface treatment marketed by Miyoshi;

a Perfluoropolymethylisopropyl ether treatment, such as the FHC surface treatment marketed by LCW;

an Isostearyl Sebacate treatment, such as the HS surface treatment marketed by Miyoshi;

a Disodium Stearoyl Glutamate treatment, such as the NAI surface treatment marketed by Miyoshi;

a Dimethicone/Disodium Stearoyl Glutamate treatment, such as the SA/NAI surface treatment marketed by Miyoshi;

a Perfluoroalkyl Phosphate treatment, such as the PF surface treatment marketed by Daito;

an Acrylate/Dimethicone Copolymer and Perfluoroalkyl Phosphate treatment, such as the FSA surface treatment marketed by Daito;

a Polymethylhydrosiloxane/Perfluoroalkyl Phosphate treatment, such as the FS01 surface treatment marketed by Daito;

a Lauroyl Lysine/Aluminium Tristearate treatment, such as the LL-StAl surface treatment marketed by Daito;

an Octyltriethylsilane treatment, such as the OTS surface treatment marketed by Daito;

an Octyltriethylsilane/Perfluoroalkyl Phosphate treatment, such as the FOTS surface treatment marketed by Daito;

an Acrylate/Dimethicone Copolymer treatment, such as the ASC surface treatment marketed by Daito;

an Isopropyl Titanium Triisostearate treatment, such as the ITT surface treatment marketed by Daito;

a Microcrystalline Cellulose and Carboxymethyl Cellulose treatment, such as the AC treatment marketed by Daito;

a Cellulose treatment, such as the C2 surface treatment marketed by Daito;

an Acrylate Copolymer treatment, such as the APD surface treatment marketed by Daito; and

a Perfluoroalkyl Phosphate/Isopropyl Titanium Triisostearate treatment, such as PF+ITT surface treatment marketed by Daito.

The pigment or pigments can be present in the composition in accordance with the disclosure in an amount ranging from 0.05 to 50% of the total weight of the composition, for example, from 0.1 to 35%.

In at least one embodiment, the composition of the present disclosure comprises at least one pigment. In at least one further embodiment, the at least one pigment is chosen from special effect pigments of mica-Fe₂O₃ type, such as Prestige Bronze.

The composition of the present disclosure can also comprise polymerization inhibitors such as anionic and/or radical polymerization inhibitors, in order to increase the stability of the composition over time. In at least one embodiment, the polymerization inhibitors are chosen from: sulphur dioxide, nitric oxide, boron trifluoride, hydroquinone and its derivatives, such as hydroquinone monoethyl ether or TBHQ, benzoquinone and its derivatives, such as duroquinone, catechol and its derivatives, such as t-butylcatechol and methoxycatechol, anisole and its derivatives, such as methoxyanisole or hydroxyanisole, pyrogallol and its derivatives, p-methoxyphenol, hydroxybutyltoluene, alkyl sulphates, alkyl sulphites, alkyl sulphones, alkyl sulphoxides, alkyl sulphides, mercaptans, 3-sulphonene and mixtures thereof. In at least one further embodiment, the alkyl groups in the polymerization inhibitors listed above possess from 1 to 6 carbon atoms.

In at least one embodiment, the polymerization inhibitor is chosen from inorganic and organic acids.

Thus, the cosmetic composition according to the disclosure can also comprise at least one inorganic or organic acid, the latter having at least one carboxyl or sulpho group, exhibiting a pKa ranging from 0 to 6, such as phosphoric acid, hydrochloric acid, nitric acid, benzene- and toluenesulphonic acid, sulphuric acid, carbonic acid, hydrofluoric acid, acetic acid, formic acid, propionic acid, benzoic acid, mono-, di- and trichloroacetic acid, salicylic acid, trifluoroacetic acid, octanoic acid, heptanoic acid and hexanoic acid.

In at least one embodiment, the polymerization inhibitor is acetic acid.

The concentration of polymerization inhibitor in the cosmetic composition of the present disclosure can range from 10 ppm to 30% by weight, for example, from 10 ppm to 15% by weight, with respect to the total weight of the composition.

The cosmetic medium of the composition of the disclosure can also comprise a specific gelling/structuring agent, such as the PSPA silicone polyamides (DP100 and DP15) sold by Dow Chemical, the polylauryldimethylsiloxane organic KSG products and the silicone KSG products sold by Shinetsu, dextrin palmitate and inulin stearate (Rheopearl range from Chiba Flour Milling), the acrylates comprising an alkyl chain sold by Landec, the ethylene/octene copolymers sold by Dupont de Nemours, the dibutyl lauroyl glutamide sold by Ajinomoto, disorbene sold by Roquette, the styrene/acrylate copolymers Versagel M5960 and 5670 sold by Penreco, the diblock and triblock Kraton products sold by Kraton Polymers, hydroxystearic acid, jojoba waxes, the pyrogenic silicas sold by Degussa, the silicone waxes sold by Waker, the polyamide Uniclear sold by Arizona Chemical, and bentone.

The composition of the disclosure can also be provided in the form of an emulsion and/or be encapsulated, the dielectrophile monomers being held in an anhydrous medium until the moment of use. When the composition of the disclosure is an emulsion, this emulsion comprises, for example, a dispersed or continuous phase, which can comprise water, C₁-C₄ aliphatic alcohols or mixtures thereof, and an anhydrous organic phase comprising a monomer. In the case of capsules or microcapsules, the capsule can comprise the monomer in an anhydrous medium and be dispersed in an anhydrous medium as defined above, water, C₁-C₄ aliphatic alcohols or mixtures thereof.

The composition of the present disclosure can also comprise at least one polymer which is not reactive with regard to the dielectrophile monomers and which is capable of increasing the viscosity of the composition. The increase in the viscosity makes it possible to reduce the rate of polymerization of the dielectrophile monomers.. To do this, it is possible to add, to the composition of the disclosure and nonexhaustively, polymethyl methacrylate (PMMA) or cyanoacrylate-based copolymers as are described in U.S. Pat. No. 6,224,622.

The composition of the disclosure can also comprise fillers. As used herein, the term “fillers,” without implied limitation, means colorless or white, inorganic or synthetic and lamellar or nonlamellar particles. They can be present in a proportion from 0 to 48% by weight, with respect to the total weight of the composition, such as from 0.01 to 30% by weight and for example, from 0.02 to 20% by weight. In at least one embodiment, fillers are chosen from talc, zinc stearate, mica, kaolin, polyamide (Nylon®) powders (Orgasol from Atochem), polyethylene powders, powders formed of tetrafluoroethylene polymers (Teflon®), starch, boron nitride, polymer microspheres, such as those of polyvinylidene chloride/acrylonitrile, for example Expancel (Nobel Industrie), or of acrylic acid copolymers (Polytrap® from Dow Corning), silicone resin microbeads (Tospearls® from Toshiba, for example), and organopolysiloxane elastomers.

The composition of the present disclosure can also comprise metal powders or particles, such as powders or particles formed of aluminium, zinc, copper, and the like.

The composition can also comprise cosmetic active principles commonly used and known in the art. Non-limiting examples of cosmetic active principles include reducing agents, oxidizing agents, fatty substances, silicones, thickening agents, softening agents, antifoaming agents, moisturizing agents, emollients, basifying agents, elastomers, plasticizers, sunscreens, direct or oxidation dyes, clays, colloidal minerals, fragrances, peptizing agents, preservatives, anionic, cationic, amphoteric, zwitterionic or nonionic surfactants, fixing or non-fixing polymers, conditioning polymers, proteins, vitamins, and the like.

The compositions according to the present disclosure can be provided in various forms, such as lotions, sprays or foams, and can be applied in the shampoo or conditioner form.

In the case of sprays, the composition of the disclosure can comprise a propellant. The propellant is comprised of the compressed or liquefied gases normally employed for the preparation of aerosol compositions. In at least one embodiment, the propellant is chosen from air, carbon dioxide gas, compressed nitrogen and a soluble gas, such as dimethyl ether, halogenated (for example, fluorinated) and nonhalogenated hydrocarbons, and mixtures thereof.

According to at least one embodiment, the presently disclosed process comprises applying, to keratinous fibers, such as the hair, at least one dielectrophile monomer and at least one radical initiator.

The radical initiator and the dielectrophile monomer can be applied simultaneously or separately. In the latter case, the initiator can be applied beforehand or vice versa.

The radical initiator can be used pure, in solution or in the form of an emulsion or can be encapsulated. It can also be added to the composition at the time of use immediately before application to keratinous fibers.

In at least one embodiment, the composition comprising at least one dielectrophile monomer and at least one radical initiator is applied to keratinous fibers, such as the hair.

If the composition comprises a dyeing means, such as a pigment, at least one embodiment of the disclosure comprises a process for dyeing by application to keratinous fibers of a composition comprising at least one dielectrophile monomer and at least one pigment.

According to at least one further embodiment of the process of the disclosure, the initiator can be applied in a stage prior or subsequent to the application of the monomer. These two components can be applied pure or in solution in a cosmetically acceptable medium. They may or may not be soluble in the solvent. One of the solvents can be a nonsolvent for the radical initiator or for the dielectrophile monomer.

In at least one embodiment, it is possible to adjust the kinetics of polymerization by moistening the fiber beforehand using an aqueous solution, the pH of which has been adjusted using a base, an acid or an acid/base mixture. The acid and/or the base can be inorganic or organic.

The process of the present disclosure can comprise additional intermediate or final stages, such as the application of a cosmetic product, a rinsing stage or a drying stage. Drying can be carried out with a hood dryer, with a hand-held hairdryer and/or with a smoothing iron. In at least one embodiment, the application of the compositions in accordance with the present disclosure can be followed by a rinsing operation.

It is also possible to carry out multiple applications of the composition of the present disclosure in order to obtain a superimposition of layers in order to try to achieve specific properties of the deposited material in terms of chemical nature, mechanical strength, thickness, appearance or feel.

In order to improve, inter alia, the adhesion of the dielectrophile monomers polymerized in situ, the fiber can be pretreated with any type of polymer.

In order to adjust the kinetics of anionic polymerization, it is also possible to increase the nucleophilicity of the fiber by chemical conversion of the keratinous fibers. For example, disulphide bridges of which the keratin is partially composed can be reduced to give thiols before application of the composition of the present disclosure. In at least one embodiment, reducing agents for the disulphide bridges of which the keratin is partially composed, are chosen from the following compounds: anhydrous sodium thiosulphate, powdered sodium metabisulphite, thiourea, ammonium sulphite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formaldehyde sulphoxylate, isooctyl thioglycolate, d,l-cysteine and monoethanolamine thioglycolate.

The application of the composition of the disclosure can also be preceded by a hair treatment, such as a direct or oxidation dyeing.

According to at least one embodiment, the dielectrophile monomers are chosen from monomers capable of polymerizing on keratinous fibers under cosmetically acceptable conditions.

In at least one embodiment, the polymerization of the dielectrophile monomer is carried out at a temperature ranging from 20 to 100° C., for example, from 20 to 90° C., which does not prevent the application being terminated by drying with a hood dryer, blow drying, subjecting to a flat iron or subjecting to a curling tong.

The disclosure further relates to a dyeing kit comprising a first compositon which comprises the dielectrophile monomer or monomers and a second composition which comprises the radical initiator or initiators.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The following nonlimiting examples make it possible to illustrate the disclosure without limiting the scope thereof.

### EXAMPLES

In the examples set forth below, locks of hair were treated with inventive compositions. After treatment, the locks were enclosed in aluminium foil and heated on a heating plate at 72° C. for 1 hour. After the aluminium foil was removed, the locks were dried with a hood dryer at 40° C. for 15 minutes. The locks were subsequently rinsed by the application of a gentle shampoo. This stage represents the initial test (to).

The dyed locks were subequently subjected to 6 shampooing operations according to a cycle which comprised wetting the locks with water, washing with the shampoos and rinsing with water, followed by drying.

The color of the locks was then evaluated sensorially at t₀ and after 6 washing operations with shampoos.

The measurements of light intensity emitted by the locks were carried out with a Minolta calorimeter (ref. CR 200).

The color of the locks were evaluated at to in the L*a*b* system. In the L*a*b* system, L* represents the intensity of the color, a* indicates the green/red color axis and b* indicates the blue/yellow color axis. The lower the value of L*, the darker the color or the more intense it is. The higher the value of a*, the redder the hue and, the higher the value of b*, the bluer the hue.

The variation in the color between the control lock composed of 90% white hairs and the lock treated with the compositions below was measured by ΔE according to the equation below from the L₀*a₀*b₀* values of the control locks and the L*a*b* values of the locks treated with the composition of the disclosure.
ΔE=\sqrt{(L*−Lₒ*)²+(a*−a₀*)²+(b*−bₒ*)²}

The greater the value of ΔE, the greater the difference in color between the control lock and the treated lock and thus the more effective the treatment.

The results are given in the tables of results below.

The saturation of the locks (SAT) was also measured. This measurement corresponds to the ratio of the colored light emitted to the white light reflected. SAT gives a report of the amount of colorant present on the lock. The higher its value, the more effective the treatment.

### Example 1

Method of Application:

The initiator was present in the composition with the dielectrophile monomer and the composition was applied to dry locks.

0.5 g of the composition below was applied to the locks of hair.

Composition

| | |
|---|---|
| Octyl 2-cyanoacrylate | 0.5 g |
| Initiator* | 0.1 g |
| Prestige Bronze | 0.5 g |
| Eckart (mica-Fe₂O₃) | |
| α, [symbol] -Dihydroxylated | 4.5 g |
| polydimethylsiloxane/cyclopentadimethylsiloxane | |
| (14.7/85.3) Dow Corning DC 1501 Fluid | |
| Acetic acid | 12 μl |

*The initiators tested are as follows:
1 - Potassium persulphate, Aldrich
2 - Ammonium persulphate, Aldrich
3 - V50 (2,2′-azobis(2-amidinopropane)dihydrochloride): Interchim ref. 759356
4 - 4,4′-Azobis(4-cyanovaleric acid), Aldrich
5 - Sodium persulphate: sold by Aldrich

*The initiators tested are as follows:
1—Potassium persulphate, Aldrich
2—Ammonium persulphate, Aldrich
3—V50 (2,2′-azobis(2-aminidinopropane)dihydrochloride): Interchim ref. 759356
4—4,4′-Azobis(4-cyanovaleric acid), Aldrich
5-Sodium persulphate: sold by Aldrich

Results

| Initiator | ΔE | SAT |
|---|---|---|
| 1 | 10.70 | 25.18 |
| 2 | 16.19 | 31.44 |
| 3 | 5.12 | 28.86 |
| 4 | 2.47 | 31.77 |
| 5 | 4.71 | 27.57 |

The locks were homogeneous, with a loose feel. The hold of the treated locks 1 to 5 after 6 shampooing operations was visually very satisfactory, the color being virtually unaffected.

### Example 2

Method of Application:

The initiator was present in the composition with the dielectrophile monomer and the composition was applied to the locks of hair immersed for 3 minutes in a buffer at pH 3 and then towel dried.

0.5 g of the composition of Example 1 was applied to the locks of hair.

Results

| Initiator | ΔE | SAT |
|---|---|---|
| 1 | 1.91 | 28.05 |
| 2 | 4.45 | 27.39 |
| 3 | 5.42 | 28.25 |
| 4 | 11.39 | 17.38 |
| 5 | 0.54 | 27.83 |

The locks were homogeneous, with a loose feel. In the same way, the hold of the treated locks 1 to 5 after 6 shampooing operations was visually very satisfactory, the color being virtually unaffected.

What is claimed is:

1. A composition comprising, in a cosmetically acceptable medium, at least one dielectrophile monomer of formula (A) and at least one radical initiator: 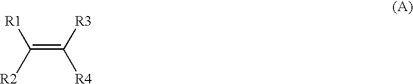
wherein:
R1 and R2 are, independently of one another, chosen from groups with little or no electron-withdrawing effect; and
R3 and R4 are, independently of one another, chosen from electron-withdrawing groups,
with the proviso that (A) cannot be methyl 2-cyanoacrylate or itaconic acid.

2. The composition according to claim 1, wherein:
R1 and R2, which are identical or different, are chosen from
i) hydrogen atoms;
ii) saturated and unsaturated, linear, branched and cyclic hydrocarbon groups, optionally comprising at least one atom chosen from nitrogen, oxygen and sulphur atoms and optionally substituted by at least one group chosen from —OR, —C(O)OR, —C(O)R, —SR and halogen atoms;
iii) modified and unmodified polyorganosiloxane residues; and
iv) polyoxyalkylene groups;
R3 and R4, which are identical or different, are chosen from —N(R)₃⁺, —S(R)₂⁺, —NO₂, —SO₂R, —C≡N, —C(O)OR, —C(O)SR, —C(O)NR₂, —F, —Cl, —Br, —I, —OR, —C(O)R and —SR groups, linear and branched alkenyl groups, linear and branched alkynyl groups, C₁-C₄ mono- and polyfluoroalkyl groups, aryl groups, and aryloxy groups;
wherein R, which is identical or different, is chosen from hydrogen atoms and saturated and unsaturated, linear, branched and cyclic hydrocarbon groups, optionally comprising at least one atom chosen from nitrogen, oxygen and sulphur atoms and optionally substituted by at least one group chosen from —OR′, —C(O)OR′, —C(O)R′, —SH, —SR′, —OH, halogen atoms, and residues of polymers which can be obtained by radical polymerization, by polycondensation and by ring opening, wherein R′ is chosen from C₁-C₁₀ alkyl groups.

3. The composition according to claim 2, wherein R1, R2 and R, which are identical or different from each other, are chosen from saturated and unsaturated, linear, branched and cyclic hydrocarbon groups comprising from 1 to 20 carbon atoms.

4. The composition according to claim 3, wherein R1, R2 and R, which are identical or different from each other, are chosen from saturated and unsaturated, linear, branched and cyclic hydrocarbon groups comprising from 1 to 10 carbon atoms.

5. The composition according to claim 2, wherein R3 and R4, which are identical or different from each other, are chosen from phenyl and phenoxyloxy.

6. The composition according to claim 1, wherein the at least one dielectrophile monomer of formula (A) is a cyanoacrylate monomer of formula (B): 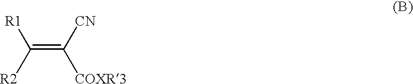
wherein
X is chosen from NH, S and O,
R′3 is chosen from hydrogen atoms, and saturated and unsaturated, linear, branched and cyclic hydrocarbon groups, optionally comprising at least one atom chosen from nitrogen, oxygen and sulphur atoms and optionally substituted by at least one group chosen from —OR′, —C(O)OR′, —C(O)R′, —SH, —SR′, —OH, halogen atoms, and residues of polymers which can be obtained by radical polymerization, by polycondensation and by ring opening, wherein R′ is chosen from C₁-C₁₀ alkyl groups,
R1 and R2 are, independently of one another, chosen from
i) hydrogen atoms;
ii) saturated and unsaturated, linear, branched and cyclic hydrocarbon groups, optionally comprising at least one atom chosen from nitrogen, oxygen and sulphur atoms and optionally substituted by at least one group chosen from —OR, —C(O)OR, —C(O)R, —SR and halogen atoms;
iii) modified and unmodified polyorganosiloxane residues; and
iv) polyoxyalkylene groups.

7. The composition according to claim 6, wherein the at least one dielectrophile monomer of formula (A) is a cyanoacrylate corresponding to the formula (C): 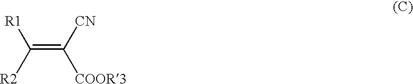
wherein
R′3 is chosen from C₁-C₁₀ alkyl radicals, C₂-C₁₀ alkenyl radicals and (C₁-C₄)alkoxy(C₁-C₁₀)alkyl radicals; and
R1 and R2 are defined as in claim 6.

8. The composition according to claim 1, wherein R1 and R2 of the at least one dielectrophile monomer of formula (A), which are identical or different, are chosen from hydrogen atoms and optionally substituted phenyl groups.

9. The composition according to claim 1, wherein the at least one dielectrophile monomer of formula (A) is an alkyl cyanoacrylate of formula (F): 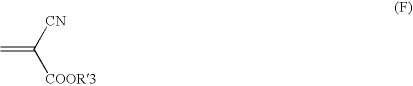
wherein R′3 is chosen from:
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

10. The composition according to claim 1, wherein the at least one dielectrophile monomer of formula (A) is chosen from (1) and (2).

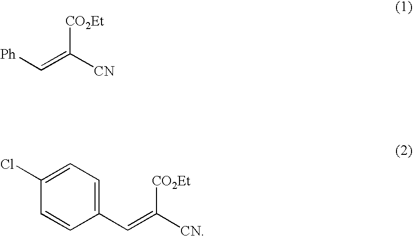

11. The composition according to claim 1, wherein the at least one dielectrophile monomer of formula (A) is present in an amount ranging from 0.1 to 80% by weight of the total weight of the composition.

12. The composition according to claim 1, wherein the at least one radical initiator is chosen from peroxides, azo compounds, persulphates, redox reagents and mixtures thereof.

13. The composition according to claim 11, wherein the at least one radical initiator is chosen from peroxides of formula (I) and (II), and mixtures thereof:
R₁—O—O—R₂ (I)
R₁—O—O—R₃—O—O—R₂ (II)
wherein:
R₁ and R₂, which are identical or different, are chosen from hydrogen atoms and from optionally substituted groups chosen from aryl, linear and branched (C₁-C₆)alkyl, acyl R′C(O)— or —C(O)R″, and ester R′OC(O)— or —(O)COR″, wherein R′ and R″, which are identical or different, are chosen from linear and branched (C₁-C₆)alkyls and optionally substituted aryl groups;
R₃ is a divalent alkylene radical —(CR₄R₅)_{q}—, wherein R₄ and R₅, which are identical or different, are chosen from hydrogen and halogen atoms, and linear and branched (C₁-C₆)alkyl groups, wherein q is an integer ranging from 1 to 6.

14. The composition according to claim 1, wherein the at least one radical initiator is present in an amount ranging from 0.01 to 40% by weight relative to the weight of the dielectrophile monomer.

15. The composition according to claim 1, further comprising at least one polymerization inhibitor.

16. The composition according to claim 1, wherein the cosmetically acceptable medium comprises at least one liquid organic solvent.

17. The composition according to claim 1, wherein the cosmetically acceptable medium comprises at least one pigment.

18. A process for the treatment of keratinous fibers comprising applying to keratinous fibers a composition comprising at least one dielectrophile monomer of formula (A) and at least one radical initiator: 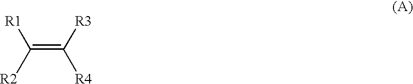
wherein:
R1 and R2 are, independently of one another, chosen from groups with little or no electron-withdrawing effect; and
R3 and R4 are, independently of one another, chosen from electron-withdrawing groups,
with the proviso that (A) cannot be methyl 2-cyanoacrylate or itaconic acid.

19. The process according to claim 18, wherein the keratinous fibers are hair.

20. A multi-compartment dyeing kit comprising a first compartment comprising a composition which comprises at least one dielectrophile monomer of formula (A): 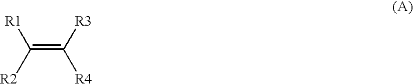
wherein:
R1 and R2 are, independently of one another, chosen from groups with little or no electron-withdrawing effect; and
R3 and R4 are, independently of one another, chosen from electron-withdrawing groups,
with the proviso that (A) cannot be methyl 2-cyanoacrylate or itaconic acid,
and another compartment comprising a second composition which comprises at least one radical initiator.

21. A process for providing keratinous fibers with lasting body, bulk and/or volume, comprising applying to said keratinous fibes a composition comprising at least one dielectrophile monomer of formula (A) and at least one radical initiator: 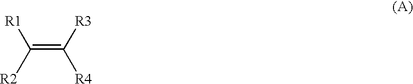
wherein:
R1 and R2 are, independently of one another, chosen from groups with little or no electron-withdrawing effect; and
R3 and R4 are, independently of one another, chosen from electron-withdrawing groups,
with the proviso that (A) cannot be methyl 2-cyanoacrylate or itaconic acid, and wherein said at least one dielectrophile monomer and said at least one radical initiator are present in the composition in a combined amount sufficient to provide lasting body, bulk and/or volume to said fibers.

22. The process according to claim 21, wherein said keratinous fibers are hair.
